(19) United States
(12) Patent Application Publication
(10) Pub. No.: US 2012/0255551 A1
(43) Pub. Date: Oct. 11, 2012

(54) RESPIRATORY ASSISTANCE DEVICE

(76) Inventor: Georges Boussignac, Antony (FR)

(21) Appl. No.: 13/434,544

(22) Filed: Mar. 29, 2012

(30) Foreign Application Priority Data
Apr. 6, 2011 (FR) 1101035

(51) Int. Cl. A61M 16/04

(52) U.S. Cl. 128/204.18

(57) ABSTRACT

The present disclosure relates to a respiratory assistance device comprising a main tube, forming a main channel, respiratory gas supplying auxiliary channels, deflection means for the respiratory gas coming from the auxiliary channels, sealing means for ensuring a sealed fluid communication between a patient's trachea and the main channel, as well as a secondary tube, forming a secondary channel, extending inside the main tube.

### CROSS-REFERENCE TO RELATED APPLICATION

This application claims priority of French Application No. 1101035, filed Apr. 6, 2011, the contents of which are expressly incorporated herein by reference.

### FIELD OF ART

The present disclosure relates to a respiratory assistance device, able to be advantageously used as an artificial respiration device while an individual under a cardiac arrest is being resuscitated.

### BACKGROUND

In particular from patent application WO2009/077667, a tubular respiratory assistance device is already known for patients whose respiration, although spontaneously occurring, is unsatisfactory. Such known respiratory assistance device comprises:
a tube forming a main channel and being intended for being connected, through its distal end, to a patient's airway so that said main channel connects to the exterior the respiratory system of the patient;
peripheral auxiliary channels connected to a source of respiratory gas, so as to blow respiratory gas in the patient's respiratory system, and opening up, via their distal end, in the main channel;
means for deflecting, towards the interior of said main channel, the respiratory gas injected through the auxiliary channels; and
sealing means, surrounding said tube, able to ensure, at the level of the patient's pharynx, a sealed fluid communication between the patient's trachea and the main channel.

Thus, in order to obtain a satisfactory respiratory gas ventilation of a patient, it is sufficient to introduce the distal end of the above mentioned device up to the pharynx of the latter (and not up to the carina through the larynx and the trachea) so as to provide the desired respiratory assistance.

Furthermore, it has been shown that such a device can be successfully used for a patient with a spontaneous respiration as an artificial respiratory device (and not only as a respiratory assistance device) on individuals under a cardiac arrest while being resuscitated, through alternate chest compressions and decompressions of their thoracic cage, the continuous jets of respiratory gas from the auxiliary channels helping to take up breath as well as bloodstream.

However, the inventor of the present disclosure has noticed that said respiratory gas, continuously introduced into the lungs of an individual under a cardiac arrest, generates therein, at the end of a compression and at the beginning of the next decompression, a positive residual pressure being maintained during a part of said decompression, before disappearing and being replaced by a negative pressure generated by the decompression. Such a positive residual pressure, on the one hand, is an obstacle to inhaling external air through said tubular member and, on the other hand, is maintained by said inhaled external air. As a result, during a large part of each decompression, said individual's lungs badly inhale the external air and the bloodstream (including the venous return) is not satisfactorily ensured at said individual's ends (head, arm, legs).

Moreover, applying compressions and decompressions on the individual's thoracic cage while being resuscitated frequently results in a loss of tightness at the level of the sealing means, being a problem, including in case of a gastric return from the esophagus. Indeed, in this latter case, the return could flow in the trachea, or even still could be introduced in the main channel of the device, resulting in an obstructive risk thereof.

### SUMMARY

The present method, system and device therefore aim at improving the known above described device, overcoming the above mentioned drawbacks.

To this end, the present disclosure is directed to a respiratory assistance device comprising:
a tube forming a main channel and being intended for being connected, through its distal end, to a patient's airway so that said main channel connects to the exterior the respiratory system of the patient;
at least one peripheral auxiliary channel being connected to a source of respiratory gas, so as to blow respiratory gas in said respiratory system, and opening up, through its distal end, in said main channel;
means for deflecting, towards the interior of said main channel, the respiratory gas injected by said auxiliary channel; and
sealing means, surrounding said main tube, able to ensure, at the level of the patient's pharynx, a sealed fluid communication between the patient's trachea and said main channel of the device,

The respiratory assistance device according to the present disclosure is remarkable in that it further comprises a secondary tube, forming a secondary channel, extending inside the main tube, over at least part of its length, and having its proximal and distal ends being intended respectively to be positioned outside the patient's mouth and to be connected to the latter's esophagus.

Thus, by means of the present method, system and device, the secondary tube produces a restriction of the main channel, increasing the resistance exerted on the gas flow circulating through the main channel and generating, upon a compression of the thoracic cage of an individual being resuscitated, a pressure increase (positive pressure) inside the lungs, the air expelled therefrom being more difficult to escape than in the absence of a secondary tube.

Conversely, upon a decompression, the pressure decreases more significantly (negative pressure) in the lungs than with a known artificial respiration device devoid of a secondary tube. Slowing down the intake of external air, generated by the diameter restriction, allows the external air to be progressively and controllably inhaled towards the individual's lungs, resulting in, at the beginning of the decompression, the positive residual pressure due to the jets of respiratory gas disappearing.

The positive residual pressure rapidly disappears under the action of the decompression, during the progressive intake of the inhaled external air. The positive residual pressure is, therefore, no longer an obstacle to inhaling external air and to the bloodstream of the individual under a cardiac arrest.

The variation of intra-thoracic pressure between a compression and a decompression, obtained according to the present method, system and device, is extended compared to the variations of intra-thoracic pressure observed on individuals being resuscitated provided with a known respiratory assistance device, for instance of the type of the one described by patent application WO2009/077667. The gas exchange interface is thereby increased and the venous return improved.

Moreover, the secondary tube allows gastric fluid to be evacuated, or even the stomach to be emptied if applicable, preventing, should the sealing means exhibit a tightness deficiency, a rejection into the trachea or the main channel of the device from being obstructed.

It should be appreciated that the respiratory assistance device of the present disclosure could be removably connected to another medical device.

The main and secondary tubes are preferably concentric over at least part of the length of said main tube.

The secondary tube could also be removably mounted with respect to said respiratory assistance device, so as to be able to be inserted in and/or removed from the main channel depending on the contemplated use.

Preferably still, said sealing means are shaped so as to ensure a sealed fluid communication between said patient's esophagus and said secondary channel.

In an embodiment according to the present method, system and device, said sealing means have the form of an inflatable bead supported by the flared and obliquely truncated periphery of a trumpet shaped part being positioned at the distal end of the main tube.

Preferably, according to this embodiment:
said secondary tube, tightly, goes through said inflatable bead and is extended outside the latter; and
said sealing means further comprise an inflatable auxiliary balloon surrounding said secondary tube, on its portion extending beyond the inflatable bead, and being able to ensure a sealed fluid communication between said patient's esophagus and said secondary channel.

In another embodiment according to the present method, system and device:
at least one communication through-hole is arranged in the side wall of the main tube, downstream the deflection means so as to allow for the fluid communication, at the level of the patient's pharynx, between his/her trachea and the main channel; and
the closed distal end of the main tube is sealingly crossed by the secondary tube.

According to this alternative embodiment, the sealing means advantageously have the shape of two distinct annular inflatable balloons, one of which surrounds the distal end of the main tube and the other one surrounds the main tube so that said communication hole is arranged between the two balloons.

Whatever the embodiment being considered, said auxiliary channel could open up in the vicinity of the proximal end of the main channel or in the vicinity of the distal end thereof.

### BRIEF DESCRIPTION OF THE FIGURES

The figures of the appended drawing will better explain how the present method, system and device can be implemented. In these figures, like reference numerals relate to like components.

FIG. 1 is a schematic and partial view, in an axial section, of an embodiment of the device of the present disclosure.

FIGS. 2 and 3 are cross-sections, respectively along the lines II-II and of the device shown in FIG. 1.

FIG. 4 is a schematic view of the sealing means of the present disclosure, according to the arrow IV of FIG. 1.

FIG. 5 schematically illustrates the positioning of the device of FIG. 1.

FIG. 6 is a schematic and partial view, in an axial section, of an alternative embodiment of the device of the present disclosure.

### DETAILED DESCRIPTION

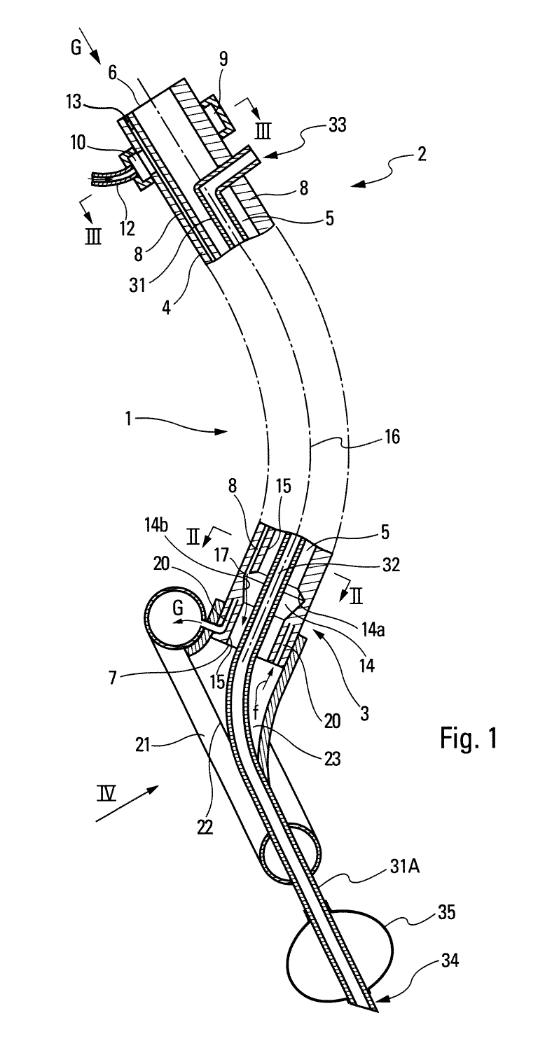

FIG. 1 schematically shows on a large scale, the sole proximal 2 and distal 3 ends of an embodiment 1 of the device according to the present disclosure.

The device 1 according to the present disclosure comprises a main flexible (or preformed, so as to conform to the patient's morphology) tube 4, delimiting a main channel 5 opening up, via the hole 6, at the proximal end 2 and, via the hole 7, at the distal end 3.

Thus, the main channel 5 is able to ensure the passage between the holes 6 and 7, one of which (the distal hole 7) is intended for being positioned inside a patient's airway, and the other one (the proximal end 6) is intended for being positioned outside said patient. This proximal hole 6 could open up in the open air and, in such a case, the patient can inhale fresh air and exhale stale air through the main channel 5 (the hole 6 could equally well be connected to a source of pressurized respiratory gas and an unidirectional valve system could be provided, so that the patient inhales the respiratory gas from said source through said main channel 5 and exhales the stale air to the open air, also through this main channel).

The diameter of the main channel 5 is of the order of a few millimetres.

Furthermore, there are arranged in the thickness of the wall of the main tube 4, auxiliary channels 8 extending over nearly the whole length of the main channel 5 and intended for being connected to a source of pressurized respiratory gas, as described hereinafter.

The connection to the source of pressurized respiratory gas could be achieved by means of a ring 9, tightly surrounding the tube 4, on the proximal end side 2, and delimiting a sealed annular chamber 10 around said tube. The auxiliary channels 8 are put in communication with the chamber 10 by means of local pulled out projections 11 of the wall of the tube 4, and said chamber 10 is connected to said source of respiratory gas by a duct 12. Obviously, the proximal ends of the channels 8 are obstructed, for instance by stoppers 13, being inserted from the proximal end face of the tube 4.

The auxiliary channels 8 have a smaller diameter than the main channel 5. The diameter of the auxiliary channels 8 is preferably lower than 1 mm and, advantageously, it is of the order of 400 to 800 microns. On the distal end, the auxiliary channels 8 open up in a recess 14 of the internal wall 15 of the tube 4. The recess 14 is annular and centered on the axis 16 of the distal end 3. It comprises a substantially transversal or slightly tilted face 14a, so as to form a flare of the main channel 5, wherein said auxiliary channels 8 open up via their holes 17, as well as a face 14b following the face 14a and converging in the direction of the axis 16.

Thus, when the auxiliary channels 8 are supplied with pressurized respiratory gas through the members 9 to 12, the corresponding gas jets hit the tilted face 14b, deflecting them in the direction of the axis 16 (see the arrow on FIG. 1 at the outlet of the holes 17), generating inside the distal end 3 of the main channel 5 an oblong shaped pressure area originating at said distal holes 17 and extending in the direction of the distal hole 7 along the axis 16 of said distal end 3. The transversal section of this pressure progressively decreases from the recess 14 to the distal hole 7, said pressure area progressively spacing apart from the internal wall 15 of the tube 4 so as to only occupy the central part of the distal end 3 thereof. Downstream the pressure area, the deflected jets of respiratory gas generate in the vicinity of the axis 16 a depression area promoting the gas circulation inside the main channel 5, from the proximal hole to the distal hole. The patient's inhalation is thereby improved.

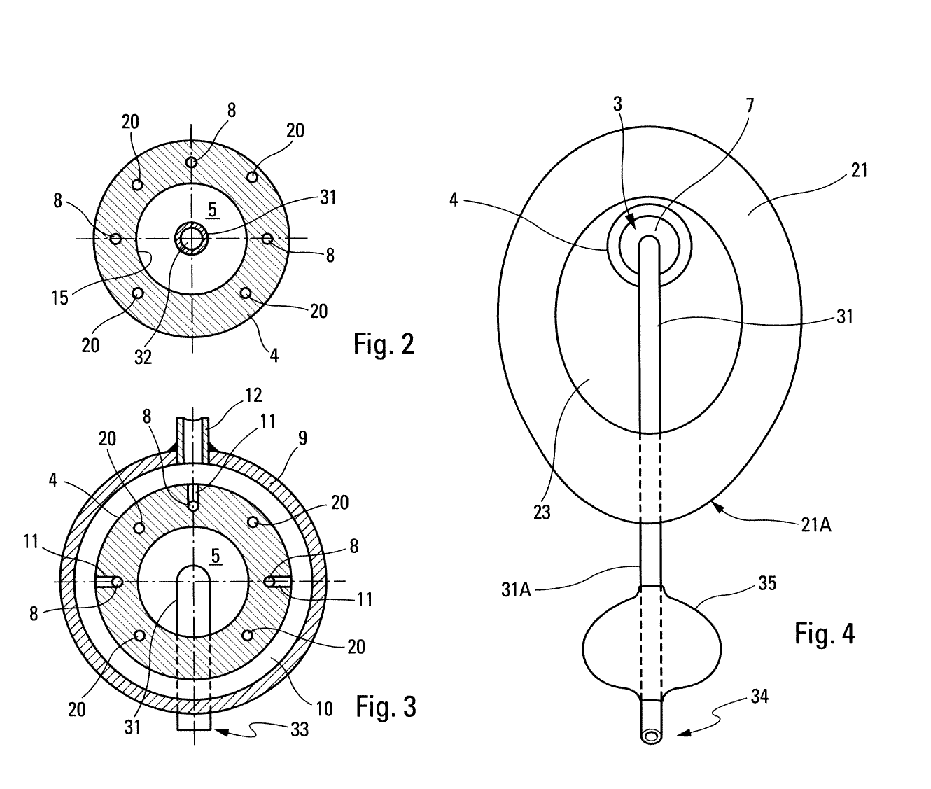

As shown on FIGS. 2 and 3, the auxiliary channels 8 are evenly arranged around the axis of the tube 4. The number thereof is variable, depending on the uses (an adult or a child), but is generally included between three and nine.

The tube 4 of the device according to the present disclosure could be made in any material already used in respiratory probes, for instance in polyvinyl chloride, with an optional silicone coating.

Additional channels 20 are provided within the thickness of said tube 4. Such channels 20 could be used for different purposes, such as injecting a fluid drug, measuring a blood pressure, taking a gas sample (as set forth symbolically by the arrow f being located facing a channel 20 at the lower part of FIG. 1) and, as will be described hereinafter, inflating a sealing bead. It should be noticed that, on FIG. 1, for drawing simplification purposes, there are shown a channel 8 and channel portions 20, although these channels are located in different plans (see FIGS. 2 and 3).

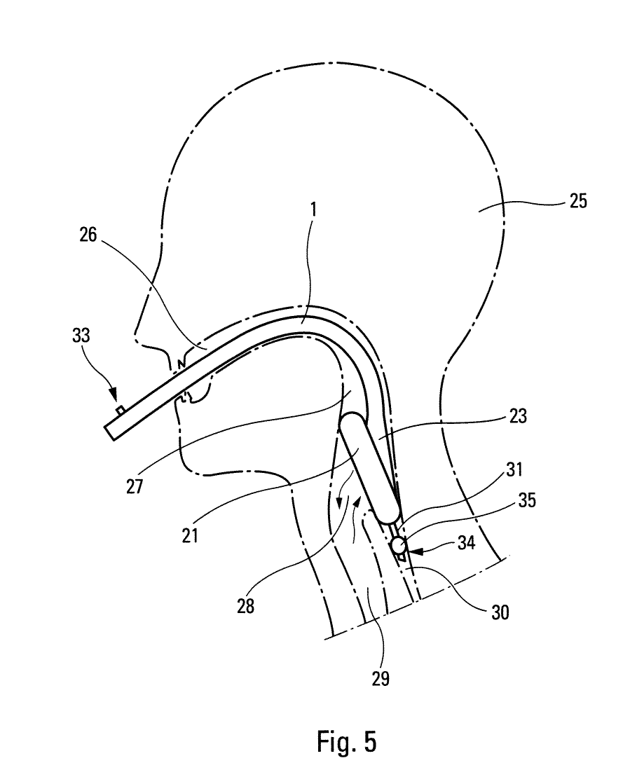

Indeed, as shown on FIGS. 1 and 4, at the distal end 3 of the tube 4, a ring-shaped inflatable bead 21 is positioned, and supported at the periphery 22 of a part 23. The part 23 has the at least approximate shape of a trumpet and is embedded via its small end on the distal end 3 of the tube 4. The flared end of the part 23 is obliquely truncated, so that said periphery 22 and the inflatable bead 21 it supports are tilted with respect to the axis 16 of the distal end 3. Thereby, when the device 1 is introduced in the deflated state into a patient 25, through his/her mouth 26 and pharynx 27, the bead 21 is able, after being inflated by an inflation gas G supplied by a (not shown) source up to the device 1, then transmitted to the bead 21 through a channel 20, to surround the larynx 28 and to provide an at least substantially sealed gas communication between the patient's 25 trachea 29 and the main channel 5 of the device 1 (see FIG. 5). In this latter position, the bead 21 partially blocks the esophagus 30 by its shaped portion 21A.

As shown on FIG. 1, according to the present method, system and device, the device 1 further comprises a flexible secondary tube 31, forming a secondary channel 32, extending inside the main tube 4, over nearly the whole length thereof. The proximal end 33 is intended for being positioned outside the patient's mouth, while the distal end 34 is, as far as it is concerned, intended for being connected to the patient's esophagus 30 as illustrated on FIG. 5.

In this example, the main tube 4 and the secondary tube 31 are concentric, the main channel 5 having an internal diameter substantially larger than the external diameter of the secondary tube 31.

Moreover, as shown on FIGS. 1 and 4, the secondary tube 31 tightly goes through the inflatable bead 21 and extends outside the latter.

The portion 31A of the tube 31, extending beyond the bead 21, comprises a ring-shaped inflatable auxiliary balloon 35, surrounding it. The latter ensures, once it is correctly positioned and inflated, a sealed fluid communication between the patient's esophagus 30 and the secondary channel 32.

An additional channel (not shown on the Figs.) is provided in the thickness of the secondary tube 31 and opens up in the auxiliary balloon 35 so as to allow an inflation gas to be injected therein.

The (not shown) lungs of the patient 25 could thereby, without the tube 4 being introduced in the trachea 29, be ventilated by means of a respiratory gas introduced in the main channel 5 through the channels 8 and the deflection means 14b and the exhalation towards the outside could be achieved of the stale gas going out of the lungs (see the two arrows on FIG. 5).

Moreover, when the distal end 34 of the secondary tube 31 is inserted in the esophagus 30 and the balloon 35 is inflated, any gastric flow is prevented in the trachea or in the pharynx, should the bead 21 exhibit a tightness defect, the tube 31 allowing for the evacuation of said flow.

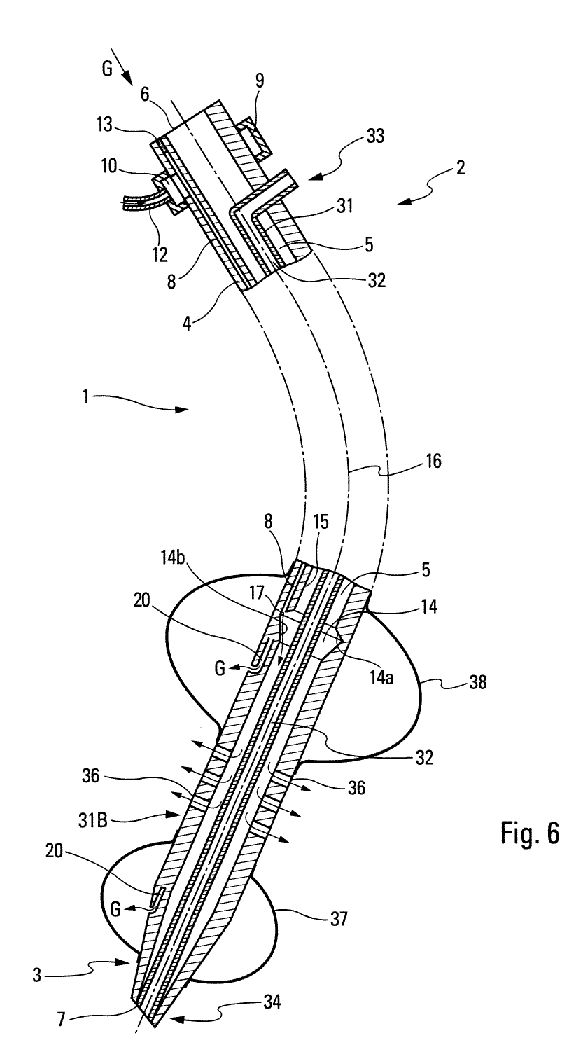

On FIG. 6, in a similar view to FIG. 1, an alternative embodiment of the device 1 according to present disclosure is shown. In the examples of FIGS. 1 and 6, like reference numerals relate to like components.

As shown on FIG. 6, the main tube 4 extends, at its distal end 3, beyond the recess 14 by a tubular portion 31B, centered on the axis 16, the side walls of which converge in the direction of this axis 16 and are integral with the external side wall of the secondary tube 31, at its distal end. In other words, in this example, the distal end 7 of the main channel 5 is tightly sealed, only the distal end 34 of the secondary channel 32 being opened so as to provide a fluid communication between the esophagus 30 and the channel 32.

Furthermore, several communication through-holes 36 are arranged in the side wall of the main tube 4, downstream the recess 14, so as to achieve the fluid communication, at the level of the patient's pharynx 27, between the trachea 29 of the latter and the main channel 5.

A first ring-shaped inflatable balloon 37 surrounds the side wall of the main tube 4, at its distal end. This balloon 37 is configured for being introduced in the patient's esophagus 30 so as to ensure a sealed fluid communication between said esophagus 30 and the secondary channel 32.

Another ring-shaped second inflatable balloon 38 surrounds the side wall of the main tube 4 so that the communication holes 36 are all intercalated between the two balloons 37 and 38. Thereby, a sealed fluid communication, at the level of the patient's pharynx 27, could be achieved between his/her trachea 29 and the main channel 5, once the two balloons 37 and 38 are inflated.

After being introduced in a deflated state through the mouth 26 and the pharynx 27 in a patient 25, the balloons 37 and 38 at position are inflated by an inflation gas G supplied from a gas source (not shown) up to the device 1, then transmitted to said balloons 37 and 38 through supplying channels 20 (partially illustrated).

1. A respiratory assistance device comprising:
a main tube forming a main channel and configured for being connected, via its distal end, to a patient's airway so that said main channel connects to the exterior of said patient's respiratory system;
at least one peripheral auxiliary channel being connected to a source of respiratory gas so as to blow respiratory gas in said respiratory system and opening up, via its distal end, in said main channel;
means for deflecting, to the interior of said main channel, the respiratory gas injected by said auxiliary channel;
sealing means for sealing fluid communication between the patient's trachea and said main channel; said sealing means surrounding said main tube, and
a secondary tube, forming a secondary channel, extending inside the main tube at least part of its length, the secondary tube comprising a proximal end and a distal end configured respectively for being arranged outside the patient's mouth and for being connected to the esophagus of the latter.

2. The device according to claim 1, wherein the main tube and the secondary tube are concentric on at least part of the length of said main tube.

3. The device according to claim 1, wherein sealing means are shaped so as to ensure a sealed fluid communication between said patient's esophagus and said secondary channel.

4. The device according to claim 1, wherein the secondary tube is removably mounted with respect to said device.

5. The device according to claim 1, wherein said sealing means have the shape of an inflatable bead supported by a flared and obliquely truncated periphery of a trumpet shaped part being positioned at the distal end of the main tube.

6. The device according to claim 5, wherein:
said secondary tube tightly goes through said inflatable bead and extends outside thereof; and
said sealing means further comprise an inflatable auxiliary balloon surrounding said secondary tube, on its portion extending beyond said inflatable bead, and configured to ensure a sealed fluid communication between said patient's esophagus and said secondary channel.

7. The device according to claim 1, wherein:
at least one communication through-hole is arranged in the side wall of the main tube, downstream the deflection means, so as to achieve the fluid communication, at the level of the patient's pharynx, between his/her trachea and the main channel; and
the closed distal end of the main tube is tightly crossed by the secondary tube.

8. The device according to claim 7, wherein the sealing means have the shape of two distinct ring-shaped inflatable balloons, and wherein one of the ring-shaped inflatable balloons surrounds the distal end of the main tube and the other one surrounds the main tube so that said communication hole is arranged between the two balloons.

9. The device according to claim 1, wherein said auxiliary channel opens up in a vicinity of the proximal end of said main channel.

10. The device according to claim 1, wherein said auxiliary channel opens up in the vicinity of the distal end of said main channel.
